(19) United States
(12) Patent Application Publication
(10) Pub. No.: US 2023/0137366 A1
(43) Pub. Date: May 4, 2023

(54) Multimodal Dialog-Based Remote Patient Monitoring of Motor Function

(71) Applicant: Modality.AI, San Francisco, CA (US)

(72) Inventors: Oliver ROESLER, Weyhe (DE); William BURKE, Portland, OR (US); Hardik KOTHARE, Burlingame, CA (US); Jackson LISCOMBE, Mill River, MA (US); Michael NEUMANN, Waiblingen (DE); Andrew CORNISH, Gore (NZ); Doug HABBERSTAD, Savanna, GA (US); David PAUTLER, San Francisco, CA (US); David SUENDERMANN-OEFT, San Francisco, CA (US); Vikram RAMANARAYANAN, San Francisco, CA (US)

(21) Appl. No.: 17/974,306

(22) Filed: Oct. 26, 2022

Related U.S. Application Data
(60) Provisional application No. 63/273,837, filed on Oct. 29, 2021.
(60) Provisional application No. 63/273,839, filed on Oct. 29, 2021.

(51) Int. Cl. A61B 5/11; G16H 40/67; G16H 50/30; A61B 5/00

(52) U.S. Cl. CPC A61B 5/1124; G16H 40/67; G16H 50/30; A61B 5/1114; A61B 5/0077; A61B 5/1121; A61B 5/4803

(57) ABSTRACT

A system and method for remote monitoring of patient motor functions includes a computing device that uses captured image data depicting a patient's body part and, based on movement information, detects whether a condition may exist that is affecting motor functions. The body part can be a hand that is tracked as the user performs a tapping exercise. The body part can also include the patient's face during speech and also without speech.

This application claims priority to U.S. provisional applications 63/273,837 and 63/273,829, both filed Oct. 29, 2021. U.S. provisional applications 63/273,837 and 63/273,829, and all other extrinsic references contained herein are incorporated by reference in their entirety.

### FIELD OF THE INVENTION

The field of the invention is patient monitoring and diagnosis.

### BACKGROUND

The background description includes information that may be useful in understanding the present invention. It is not an admission that any of the information provided herein is prior art or relevant to the presently claimed invention, or that any publication specifically or implicitly referenced is prior art.

The need for remote monitoring to support Parkinson's Disease patients, caregivers, and healthcare professionals in their collaborative efforts for better care has never been greater. This situation has been brought into greater focus by the COVID-19 pandemic, which made it more difficult for patients to safely visit with their doctors and other care providers.

Existing solutions attempt to measure a user's motor functions by requiring a user to conduct finger-tapping exercise on a screen. In these solutions, the user taps buttons as prompted on a screen. For example, the paper “Detecting and monitoring the symptoms of Parkinson's disease using smartphones: a pilot study” published in 2015 discusses such an approach.

Unfortunately these solutions suffer from multiple limitations. Having a user tap on a screen can affect the results of the test because the size of the screen used for the test can vary. Moreover, this sort of test only measures tapping, not the motion in between the taps. Additionally, this type of test lacks the ability to tell whether a user is properly conducting the test. Finally, this approach can only use tapping information for its conclusions.

Thus, there is still a need for a multi-modal test that allows for accurate remote monitoring for patients.

### SUMMARY OF THE INVENTION

The inventive subject matter provides apparatus, systems and methods in which a computing device scans a body part of a user and maps two points on that body part. The two points are then used by the computing device to detect a relative movement between the body parts. The detected movement is compared by the computing device against one or more metrics. The result is then used by the computing device to determine the existence of condition.

In embodiments of the inventive subject matter, the body part used is the user's hand and the points are a point on the thumb and a point on an index finger.

In embodiments of the inventive subject matter, the body part used is the user's face and the points are points at one or more facial features.

The computing device can track relative movement between two points as a part of a tapping exercise. The computing device can detect the distance in the relative movement and the speed (e.g., tapping time), as well as a consistency over a period of time.

In embodiments, the computing device can deploy a virtual agent that guides the patient through conversation. The patient's speech is captured and analyzed to determine whether a condition exists.

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 is a diagrammatic overview of a system that executes processes associated with embodiments of the inventive subject matter.

FIG. 2 provides a flowchart of the recognition and diagnosis processes executed according to the systems and methods of the inventive subject matter.

FIGS. 3A and 3B show an illustration of a user's hand with mapped points at an open state and a closed state, respectively, according to embodiments of the inventive subject matter.

FIG. 4 provides a flowchart of the conversational processes executed by the system, according to embodiments of the inventive subject matter.

FIG. 5 is a flowchart of a process to analyze image data of a patient's face, according to embodiments of the inventive subject matter.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

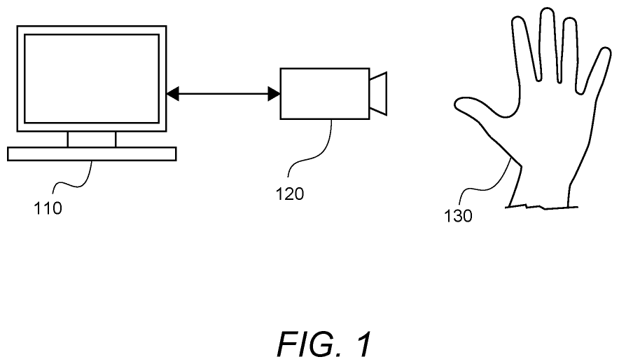

FIG. 1 provides a diagrammatic overview of a system 100 that executes the processes discussed herein, according to embodiments of the inventive subject matter.

The system 100 includes a computing device 110 that is communicatively coupled with a sensor 120. In the embodiments shown herein, sensor 120 is considered to be an image sensor (e.g., a camera) that is capable of capturing images of a body part 130 (in this example, a user's hand). However, it is contemplated that other sensors 120 could be used. For example, for embodiments that involve a patient speaking as a part of the evaluation, the sensor 120 can include a microphone.

The computing device 110 can include the camera 120 or can be separate from the camera 120. Suitable computing devices can include smartphones, tablets, desktop computers, laptop computers, gaming consoles, etc.

In embodiments, the computing device 110 is local to the user. In these embodiments, the computing device 110 can obtain information and execution code from a remote server in order to carry out the processes of the inventive subject matter locally.

In embodiments, the camera 120 can be local to the user (such as a standalone camera with data-exchange capability, or a camera within a local computing device) and computing device 110 can be remote from the user (e.g., a remote server) and connected to the device of camera 120 via a data exchange network such as the internet. In these embodiments some or all of the processes associated with the computing device 110 can be performed remotely. For example, in some embodiments, the remote computing device 110 performs all of the processes and the local computing device with camera 120 is only used for image capture and other user interactions. In a variation of these embodiments, some of the processes can be carried out locally by a local computing device while others are carried out remotely by a remote computing device, thus distributing the computing load.

The image data captured by the camera 120 is preferably video image data, though a series of still images can also be used.

It should be noted that in the embodiments discussed herein, the patient can be (and often is), remotely located from any health care provider—it could be their home, which may be geographically distant from their health care provider.

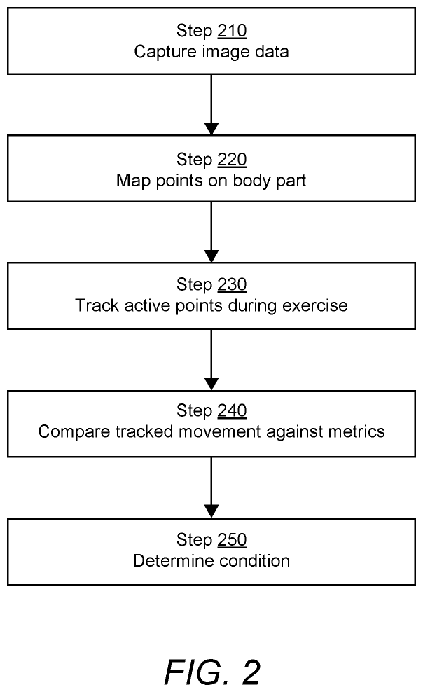

FIG. 2 provides a flowchart of the recognition and diagnosis processes executed according to the systems and methods of the inventive subject matter.

Prior to the start of the processes discussed herein, the computing device 110 can be programmed to detect the presence of necessary hardware (e.g., a camera 120, microphone, etc.) and can conduct tests of the camera, microphone, speaker, etc. that will be used by the patient. The tests of these devices can include tests to determine that the devices are providing sensor data of a sufficient quality (e.g., proper microphone sensitivity, sufficient video resolution and frame rate, etc.) for the tests discussed here. A patient can access the functions of the system via a weblink, login portal, or other known methods of accessing networked or distributed computer systems.

At step 210, a camera 120 captures image data of a part of a patient's body that is to be used for the test. In this example, the image data depicts the patient's hand 130.

By applying image recognition techniques, the computing device 110 recognizes the body part in the image data. In embodiments, the computing device 110 can provide instruction if it detects that the body part is not fully within the image or the image is otherwise unusable (e.g., glare, unfocused, etc.). Thus, for example, if the hand is too close to the camera 120 such that the relevant portions of the hand are not fully visible, the computing device 110 displays instructions to the user to move the hand away.

At step 220, the computing device maps points on the body part 130 based on the captured image. The points include two active points that are to be used to determine a relative movement between the points for the purposes of the patient test.

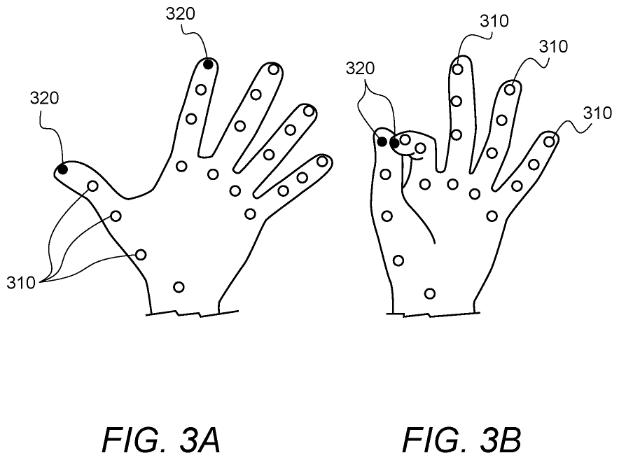

FIGS. 3A and 3B show an example of a user's hand at fingers open state (FIG. 3A) and fingers closed state (FIG. 3B) with points 310. In this example, the active points 320 are those at the end of the thumb and the pointer finger in this example. The current example shows two active points 320. However, it is contemplated that more than two active points 320 can be detected and used, depending on the exercise to be performed, the body part to be analyzed/tracked, the precision necessary to perform measurements, and other factors.

In embodiments the computing device maps the points by recognizing the body part (e.g., the hand of FIGS. 3A-3B) at step 210 and then superimposing points digitally over the captured body part using image recognition techniques.

In other embodiments, the points are physically marked on the patient's body part (such as with a marker). These physical marks then appear in the image data of the body part and are detected by the computing device at step 220.

In embodiments, software such as MediaPipe Hands can be used for hand and hand landmark (i.e., the points 310, 320) detection.

At step 230, the computing device 110 tracks the movement of the active points 320 as the user as the user performs an exercise.

The exercises such as the tapping exercise can be known exercises used for the diagnosis of conditions. For example, the tapping exercises can be those of section 3.4 of the Movement Disorder Society-Unified Parkinson's Disease Rating Scale (“MDS-UPDRS”). In this case, the exercise is a tapping exercise that requires the user to tap their thumb and pointer finger together (FIG. 3B) and then spread them apart as much as and as fast as they can (FIG. 3A).

In embodiments of the inventive subject matter, the computing device 110 can display prompts or instructions that show a user how to perform the exercise.

The tracking of the movements can include timing each of the individual taps during the exercise, measuring the lengths of the range of motion of each of the taps.

The computing device 110 can also track for interruptions or pauses during the exercise by determining the change in position on a frame-by-frame basis and detecting a pause by determining that the position of the fingers has not changed for a certain number of frame after being in motion.

In embodiments of the inventive subject matter, the computing device 110 tracks the movement to determine that the patient is performing the exercise correctly. If the computing device 110 determines that the patient is not performing the exercise correctly, it can provide feedback by way of instructions to assist the patient in correcting the way they are performing the exercise. To do so, the computing device 110 can compare the patient's captured movement against templates of tracked movements to determine a similarity within a percentage threshold. For example, based on the tracking of the active points 320 as well as other points 310 on the hand, the computing device 110 may determine that they user is not fully extending their finger and thumb during the exercise. In another example, tracking the points 320, 310 can allow the computing device 110 to determine that the patient is using the wrong finger to tap with the thumb.

The instructions provided by the computing device 110 can include textual instructions displayed on a screen, audio instructions, and/or a video or animation that illustrates the correct way of performing the exercise.

At step 240, the computing device 110 compares the tracked movement of the active points 320 against one or more baseline metrics. The baseline metrics are metrics that can correspond to the movements of a person having normal or unaffected motor functions.

The baseline metrics can include speed metrics (i.e., that set a baseline about how quickly the full range of motion of a single repetition of finger tapping exercise should take), a range of motion metric (i.e., that sets a baseline regarding the range of motion between the point at which the thumb and finger touch and the point of the motion when they are farthest apart), and consistency metrics (i.e., that set a baseline regarding the consistency of a speed and/or range of motion of each tap during the entire tapping exercise).

In embodiments, the baseline metrics can be set based on historical data from the user such that a baseline for that particular user can be set. In other embodiments, the baseline metrics can be set based on the first tap or first set of taps performed during the exercise (i.e., reflecting a “rested” condition on the part of the user) and the subsequent taps compared against these baseline taps.

Thus, for finger tapping exercises that ask the user to tap as quickly as they can, the tracked motion is compared against speed metrics; for finger tapping exercises that as the user to make the tapping movement as wide as possible, the tracked motion is compared against range of motion metrics, etc.

The consistency metrics can include a slowing down of the pace of the finger taps from one tap to the next across the exercise.

In embodiments where the system 100 implements the tests from section 3.4 of the MDS-UPDRS, the computing device 110 tracks the regularity and smoothness of the rhythm during the tapping exercise (e.g., interruptions or hesitations), the slowing of the pace during the exercise and a change in the amplitude (the range of motion between the fully opened hand and fingers touching, and back) of the movements after the start. The metrics used in these embodiments can include the number of interruptions, the amount of slowing of the pace, or the decrease in amplitude after a certain number of repetitions. To do so, the computing device 110 determines a maximum distance, a maximum velocity and a maximum acceleration across all of the cycles (taps) during the exercise, a difference between the average velocity and acceleration during the first and second half of the exercise, a “jitter” (a cycle-to-cycle variation of the time period), and a “shimmer” (a cycle-to-cycle variation of the amplitude).

At step 250, the computing device determines the existence of a condition based on the comparison of the tracked movement against the one or more metrics of step 240.

The comparison at step 240 can be against more than one metric such that the combination of results are used to determine the patient's condition.

In embodiments that use the MDS-UPDRS, the comparison of the performance of the patient during the test against the metrics is scored. The score can then be used as a part of an assessment to determine whether the patient has a condition (or likely has a condition) and the severity of the condition.

In embodiments of the inventive subject matter, the computing device 110 is also programmed to execute a virtual dialog agent that engages with a patient to elicit certain speech and facial behaviors. The virtual dialog agent provides instructions to the user such that the patient responds in manner that enables the computing device to detect and analyze the speech in accordance with section 3.1 of the MDS-UPDRS.

The condition detected can include motor function disorders, neurological disorders, Parkinson's disease, or other conditions.

In embodiments, step 250 can also include gathering and delivering diagnostic data to a health care provider. In these embodiments, the computing device 110 gathers data associated with the performance of the test by the patient. For example, for a finger tapping exercise, the diagnostic data could include measured amplitude and/or speed during the exercise. The diagnostic data could also/instead include measured changes in the detected movement of the patient's body part during the test. For example, a decrease in the amplitude or a slow-down in the speed as the test progresses.

Once the computing device 110 has gathered the data from the test(s), it can transmit the data to the computing device(s) of one or more health care providers. As mentioned herein, these providers may be geographically remote from the patient.

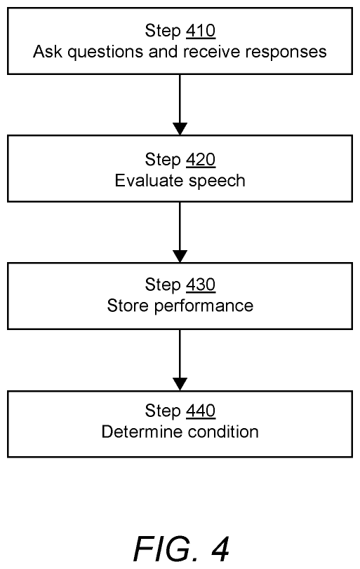

FIG. 4 provides a flowchart of the conversational processes executed by the system 100, according to embodiments of the inventive subject matter.

To do so, the computing device 110 (via the virtual dialog agent), asks the patient questions and then, via the camera 120 (that includes a microphone), captures the response at step 410. The questions are typically open ended to elicit spoken responses that extend beyond “yes” or “no” responses.

The computing device 110 then evaluates the speech according to one or more metrics at step 420. The metrics used in the evaluation of the speech include volume, modulation (prosody), and clarity. Clarity metrics can include detecting slurring, palilalia (repetition of syllables) and tachyphemia (rapid speech, running syllables together).

To evaluate the speech, the computing device 110 employs speech recognition software. For example, the speech recognition software can transcribe what it “understands” and then compare that against known words and phrases to determine the level of correct or accurate understanding. The speech recognition software can also detect the repetition of syllables and the speech speed and cadence.

At step 430, the computing device 110 scores the evaluated speech against a scoring metric. One such scoring metric is in section 3.1 of the MDS-UPDRS, which assigns point values based on the patient's modulation, diction, volume and ease of understanding.

Based on the scoring of the speech, the computing device 110 then determines a possible condition at step 440.

It is contemplated that the processes of FIG. 4 can be executed prior to, concurrently with or after the processes of FIG. 2.

A technique for the collection and use of speech in the determination of conditions that could be applied to the methods and systems discussed herein is discussed in Applicant's own provisional application 63/273,829 titled “On the robust automatic computation of speaking and articulation duration in ALS patients versus healthy controls”, incorporated by reference in its entirety.

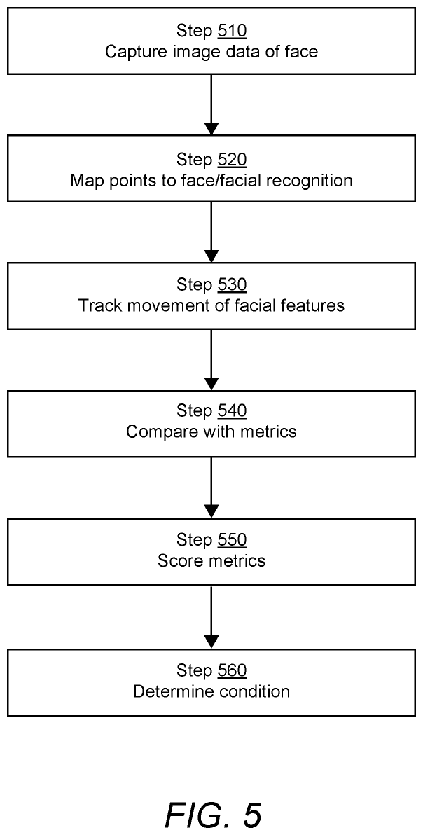

In embodiments of the inventive subject matter, the computing device 110 is also programmed to analyze image data of the patient's face in accordance with section 3.2 of the MDS-UPDRS. FIG. 5 shows this process.

To do so, the computing device 110 receives image data (video image data or a series of still images) from the camera 120 that is capturing the patient's face at step 510.

At step 520, the computing device 110 can map points to the user's face (similar to that of step 220 with the hand). Unlike the hand example of FIGS. 2 and 3A-3B, the computing device 110 uses active points at a number of facial features. For example, at the top and bottom lips of the patient to calculate relative movement between the lips and the speed thereof or at the top lip and at a point on the jaw, at the eyelids and under the eyes to detect blinking frequency, at the corners of the mouth to detect smiling, etc.

In other embodiments, facial recognition software can be used that can detect facial features and their movements.

At step 530, the computing device 110 tracks the movement of the various facial features during the exercise. The exercise can involve talking as well as without talking.

Based on the detected movements of the various facial features, the computing device 110 analyzes the movements to determine metrics at step 540. The metrics can include eye-blink frequency, makes facies or loss of facial expression, smiling, and parting of lips. Thus, the computing device 110 determines the amount of blinks/blink frequency, changes in facial expression, smiling, parting of lips, etc. based on the movement of the facial features.

At step 550, the metrics are scored by the computing device 110. An example of how the scoring can occur is included in MDS-UPDRS section 3.1.

Based on the scoring, the computing device 110 determines a condition or possible condition at step 560.

The processes of FIGS. 2, 4 and 5 can be used separately, but in preferred embodiments they are used together as part of a complete evaluation to enhance the accuracy of the condition determination.

In embodiments of the inventive subject matter, the computing device 110 is programmed to detect correlations between the tracked movements and speech of the different tests of FIGS. 2, 4 and 5, and report the correlations. These correlations can also be used as a part of a determination of a condition. For example, the speech test and the finger-tapping test can be performed simultaneously. In these situations, the computing device 110 can determine correlations between pauses or interruptions in the flow of the finger-tap exercise and pauses or interruptions in speech. Moreover, the computing device 110 can determine which of the pauses or interruptions are leading or lagging.

In the embodiments shown herein, a camera is used as the primary sensor for the purposes of the processes discussed herein. However, other sensors can be used instead of or in addition to a camera. Other sensors that could be used include an infrared sensor, a stand-alone microphone (for tests that use speech), wearable sensors (e.g., gloves with sensors at the fingertips capable of detecting movement and speed), and other sensor.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for remote patient monitoring of motor functions, comprising:
scanning, by a computing device, a body part of a user;
mapping, by the computing device, at least two points on the body part of a user;
detecting, by the computing device, a relative movement between the at least two points;
comparing, by the computing device, the detected relative movement against at least one metric; and
determining, by the computing device, the existence of a condition based on the comparison of the detected relative movement against the at least one metric.

2. The method of claim 1, further comprising wherein:
the body part comprises a hand of the user; and
the at least two points comprise a point on a thumb on the hand and a point on a finger on the hand.

3. The method of claim 2, wherein the relative movement comprises a movement of the point on the thumb relative to the point on the finger of the hand during the execution of a tapping exercise.

4. The method of claim 3, wherein detecting the relative movement further comprises detecting at least one of a relative movement distance and a tapping time during the execution of the tapping exercise.

5. The method of claim 1, wherein the scanning by a computing device further comprises:
capturing, by an image sensor, image data that includes an image of the body part; and
recognizing, by the computing device using image recognition, the body part as a usable body part.

6. The method of claim 5, wherein the image data comprises video data, and wherein the image sensor is located remotely from a care provider.

7. The method of claim 1, wherein the detected relative movement comprises a repeated relative movement and the at least one metric comprises a decrease in a speed of movement or of a range of movement during the repeated relative movement.

8. The method of claim 7, wherein the condition comprises a disorder of motor function.

9. The method of claim 1, wherein the body part of a user comprises a user's face and the at least two points on the body part comprise a point on a lip of the user and a point on the jaw of the user.

10. The method of claim 1, further comprising:
recording, by an audio capture device, speech audio from the user;
comparing, by the computing device, at least one speech characteristic against a speech metric;
determining, by the computing device, the existence of the condition based on the comparison of the at least one speech characteristic against the speech metric and the comparison of the detected relative movement against the at least one metric.

11. The method of claim 10, wherein the at least one speech characteristic comprises at least one of a speaking rate or a speaking duration.

12. The method of claim 1, wherein the step of determining the existence of a condition further comprises:
gathering diagnostic data; and
delivering the diagnostic data to a care provider.

13. The method of claim 12, wherein the diagnostic data comprises a measured change in the detected relative movement across multiple repetitions.

14. The method of claim 12, wherein the diagnostic data comprises at least one of a measured amplitude and speed across a tapping exercise.

15. The method of claim 1, wherein the scanning is performed via at least one of a video camera, an infrared sensor, and a wearable sensor.
